(19) United States
(12) Patent Application Publication
(10) Pub. No.: US 2022/0285038 A1
(43) Pub. Date: Sep. 8, 2022

(54) PREDICTION METHOD, ELECTRONIC DEVICE, AND STORAGE MEDIUM

(71) Applicant: BEIJING SENSETIME TECHNOLOGY DEVELOPMENT CO., LTD., Beijing (CN)

(72) Inventors: Qiao LIU, Beijing (CN); Zhiqiang HU, Beijing (CN)

(21) Appl. No.: 17/739,541

(22) Filed: May 9, 2022

Related U.S. Application Data
(63) Continuation of application No. PCT/CN2020/103633, filed on Jul. 22, 2020.

(30) Foreign Application Priority Data
Nov. 18, 2019 (CN) 201911125921.X

(51) Int. Cl. G16H 70/40; G16H 20/10; G06N 3/08

(52) U.S. Cl. CPC G16H 70/40; G16H 20/10; G06N 3/08

(57) ABSTRACT

A prediction method, an electronic device and a storage medium are provided. The method includes that: substance features of a substance to be tested are determined according to a molecular structure of the substance to be tested; feature extraction is performed on a diseased cell of a target category to obtain at least one cell feature of the diseased cell; and a response result of the substance to be tested against the diseased cell is predicted according to the substance features and the at least one cell feature.

### CROSS-REFERENCE TO RELATED APPLICATION

The present disclosure is a continuation of International Application No. PCT/CN2020/103633, filed on Jul. 22, 2020, which is based upon and claims priority to Chinese Patent Application No. 201911125921.X, filed on Nov.18, 2019. The contents of International Application No. PCT/CN2020/103633 and Chinese Patent Application No. 201911125921.X are hereby incorporated by reference in their entireties.

### BACKGROUND

Due to the uncertainty of drug efficacy and the heterogeneity of cancer patients, it is important to accurately test whether drugs have an inhibitory effect on the cancer cells.

In related arts, a machine learning is generally performed based on drug features (such as molecular fingerprints) extracted manually and cancer cell features extracted from single omics data of cancer cells, to obtain an inhibitory effect of the drug on this type of the cancer cells. The drug features extracted manually are often sparse, so the final inhibitory effect is less accurate and the calculation process is relatively inefficient.

### SUMMARY

The present disclosure relates to the field of computer technologies, and the embodiments of the present disclosure propose a prediction method, an electronic device, and a storage medium.

According to a first aspect of the present disclosure, there is provided a prediction method, including the following operations.

According to a molecular structure of a substance to be tested, substance features of the substance to be tested are determined.

Feature extraction is performed on a diseased cell of a target category to obtain at least one cell feature of the diseased cell.

According to the substance features and the at least one cell feature, a response result of the substance to be tested against the diseased cell is predicted.

According to a second aspect of the present disclosure, there is provided an electronic device, including a processor and a memory configured to store instructions that, when executed by the processor, cause the processor to perform the following operations.

According to a molecular structure of a substance to be tested, substance features of the substance to be tested are determined.

Feature extraction is performed on a diseased cell of a target category to obtain at least one cell feature of the diseased cell.

According to the substance features and the at least one cell feature, a response result of the substance to be tested against the diseased cell is predicted.

According to a third aspect of the present disclosure, there is provided a non-transitory computer-readable storage medium having stored thereon computer program instructions that, when executed by a processor of an electronic device, cause the processor to perform the prediction method according to the first aspect.

It should be understood that the above general description and the following detailed description are only exemplary and explanatory, rather than limiting the present disclosure. According to the following detailed description of exemplary embodiments with reference to the accompanying drawings, other features and aspects of the present disclosure will become clear.

### BRIEF DESCRIPTION OF THE DRAWINGS

The drawings herein are incorporated into the specification and constitute a part of the specification. These drawings illustrate embodiments that conform to the present disclosure, and are used together with the specification to explain the technical solutions of the present disclosure.

FIG. 1 is a flowchart of a prediction method provided by an embodiment of the present disclosure.

FIG. 2 is a diagram of a matrix provided by an embodiment of the present disclosure.

FIG. 3 is a flowchart of a prediction method provided by an embodiment of the present disclosure.

FIG. 4 is a structural diagram of a prediction device provided by an embodiment of the present disclosure.

FIG. 5 is a structural diagram of an electronic device provided by an embodiment of the present disclosure.

FIG. 6 is a structural diagram of an electronic device provided by an embodiment of the present disclosure.

### DETAILED DESCRIPTION

Various exemplary embodiments, features, and aspects of the present disclosure will be described in detail below with reference to the drawings. The same reference numerals in the drawings indicate elements with the same or similar functions. Although various aspects of the embodiments are shown in the drawings, unless otherwise noted, the drawings are not necessarily drawn to scale.

The dedicated word “exemplary” herein means “serving as an example, embodiment, or illustration”. Any embodiment described herein as “exemplary” need not to be construed as being superior or better than other embodiments.

The term “and/or” herein is only an association relationship describing associated objects, which means that there will be three relationships. For example “A and/or B” has three meanings: A exists alone, A and B exist at the same time and B exists alone. In addition, the term “at least one” herein means any one of the multiple or any combination of at least two of the multiple. For example, including at least one of A, B or C means including any one or more elements selected from a set formed by A, B and C.

In addition, in order to better explain the present disclosure, numerous specific details are given in the following detailed embodiments. Those skilled in the art should understand that without certain specific details, the present disclosure are also implemented. In some embodiments, the methods, means, elements, and circuits well known to those skilled in the art have not been described in detail, so as to highlight the gist of the present disclosure.

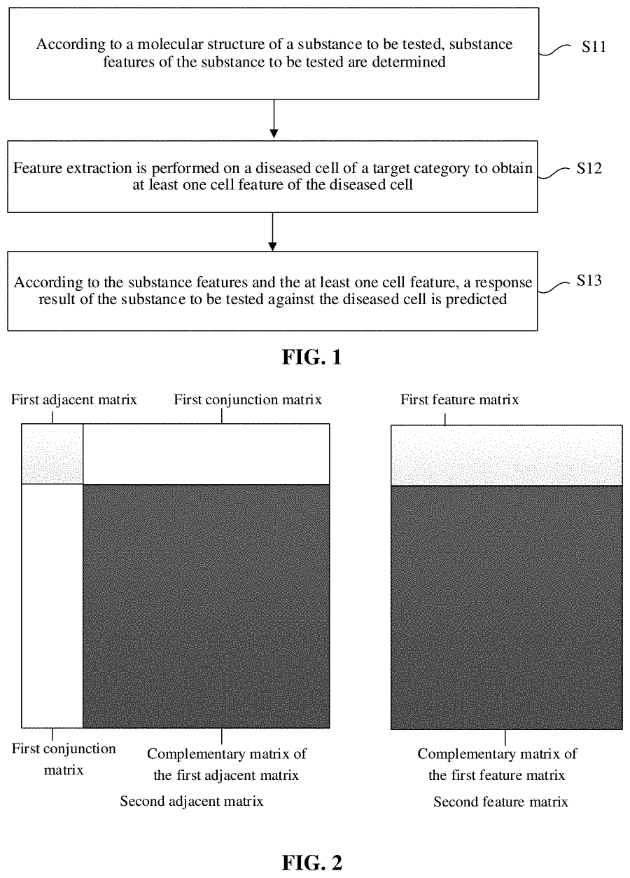

FIG. 1 is a flowchart of a prediction method provided by an embodiment of the present disclosure. The prediction method is performed by a terminal device or other processing devices. The terminal device is user equipment (UE), a mobile device, a user terminal, a terminal, a cellular phone, a cordless phone, a personal digital assistant (PDA), a handheld device, a computing device, a vehicle-mounted device, a wearable device, etc. Other processing devices are servers or cloud servers. In some possible implementations, the prediction method is implemented by a processor through invoking computer-readable instructions stored in a memory.

As shown in FIG. 1, the prediction method includes the following operations.

In S11, according to a molecular structure of a substance to be tested, substance features of the substance to be tested are determined.

For example, the substance to be tested is a substance with the molecular structure, such as a drug. The molecular structure of the substance to be tested is composed of multiple atoms and atomic bonds between the multiple atoms, and the substance features of the substance to be tested are extracted according to the molecular structure of the substance to be tested.

In a possible implementation, the substance features of the substance to be tested are determined according to the molecular structure of the substance to be tested, which includes that: a structure feature map of the substance to be tested is constructed according to the molecular structure of the substance to be tested, herein, the structure feature map includes at least two nodes and lines between the nodes, each node represents an atom in the molecular structure, and each line represents an atomic bond in the molecular structure; and according to the structure feature map, the substance features of the substance to be tested are determined.

For example, according to the molecular structure of the substance to be tested, a structure feature map of the substance to be tested is constructed. The molecular structure of the substance to be tested is composed of at least two atoms and atomic bonds between the at least two atoms. Thus, the structure feature map of the substance to be tested includes at least two nodes and lines between the nodes. Here, each node represents an atom in the molecular structure, and each line between the nodes represents an atomic bond between the atoms.

The substance features of the substance to be tested are obtained by performing feature extraction on the structure feature map of the substance to be tested. Exemplarily, a convolutional neural network, that performs feature extraction on a structure feature map, is pre-trained and is used to perform feature extraction on the structure feature map of the substance to be tested to obtain the substance features of the substance to be tested. In such a way, the substance features of the substance to be tested are extracted based on the structure feature map of the substance to be tested, and the substance features extracted in this way are denser than the substance features extracted manually. Furthermore, by performing the prediction based on the substance features, the accuracy of the test result and the efficiency of obtaining the test result will be improved.

In S12, feature extraction is performed on a diseased cell of a target category to obtain at least one cell feature of the diseased cell.

For example, the target category is a certain cancer or any other types of lesions, which is not limited in the present disclosure. Exemplarily, at present, a therapeutic drug B for A-type cancer is developed, and it is necessary to test the response of drug B to the cancer cell of the A-type cancer, thus the drug B is called the substance to be tested, and the cancer cell of the A-type cancer is called the diseased cell of a target category.

Exemplarily, a convolutional neural network, that performs feature extraction on the diseased cell, is pre-trained and is used to perform cell feature extraction on the diseased cell to obtain at least one cell feature of the diseased cell. For example, at least one of a genome feature, a transcriptome feature, or an epigenome feature of the diseased cell is extracted.

In S13, according to the substance features and the at least one cell feature, a response result of the substance to be tested against the diseased cell is predicted.

After the substance features of the substance to be tested and the at least one cell feature of the diseased cell are obtained, a prediction operation can be performed according to the substance features of the substance to be tested and the at least one cell feature of the diseased cell to obtain the predicted response result of the substance to be tested against the disease cell.

Exemplarily, a convolutional neural network, that performs a response prediction according to the substance features and at least one cell feature, is pre-trained and is used to perform a prediction operation on the substance features of the substance to be tested and the at least one cell feature of the diseased cell to obtain the predicted response result of the substance to be tested against the diseased cell.

In a possible implementation, the response result of the substance to be tested against the diseased cell is predicted according to the substance features and the at least one cell feature, which includes that: the substance features and the at least one cell feature are concatenated to obtain a combined feature; and convolution processing is performed on the combined feature to obtain the predicted response result of the substance to be tested against the diseased cell.

For example, a combined feature is obtained by directly concatenating the substance features of the substance to be tested and the at least one cell feature. The combined feature is represented as: substance feature +cell feature. The convolution processing is performed on the combined feature through the pre-trained convolutional neural network that performs the response test. The output of the convolutional neural network is a probability value between 0 and 1, herein, the probability value indicates a probability that the substance to be tested plays an inhibitory role on the diseased cell.

In this way, according to the molecular structure of the substance to be tested, the substance features of the substance to be tested are determined, and the at least one cell feature of the diseased cell of the target category is extracted, and then the response result of the substance to be tested against the diseased cell is predicted according to the substance features of the substance to be tested and the at least one cell feature of the diseased cell. According to the prediction method provided by the embodiments of the present disclosure, the substance features of the substance to be tested are extracted based on the molecular structure of the substance to be tested, and the substance features extracted in this way are denser than the substance features extracted manually. When the extracted substance features are adopted to predict the response result, the test accuracy of the response result and the efficiency of obtaining the test result are improved.

In a possible implementation, the substance features of the substance to be tested are determined according to the structure feature map, which includes that: according to the structure feature map, a first adjacent matrix and a first feature matrix of the substance to be tested are obtained, herein, the first adjacent matrix represents neighbor relationships between atoms of the substance to be tested, and the first feature matrix represents attribute data of each atom of the substance to be tested; and according to the first adjacent matrix and the first feature matrix of the substance to be tested, the substance features of the substance to be tested are obtained.

For example, the neighboring atoms of each atom of the substance to be tested are extracted according to the structure feature map, and a first adjacent matrix is formed according to the neighboring atoms of each atom, and each row of the first adjacent matrix represents the neighbor relationships between an atom of the substance to be tested and other atoms, herein, the neighbor relationships refer to connection relationships. For example, the first row of the first adjacent matrix indicates whether the first atom of the substance to be tested has connection relationships with other atoms, if the first atom has an connection relationship with one of other atoms, it is represented as 1 in the first adjacent matrix, otherwise, it is represented as 0 in the first adjacent matrix. Each atom of the substance to be tested is extracted according to the structure feature map, and attribute data of each atom is obtained. For example, the attribute data of each atom is queried from a database. The attribute data includes, but is not limited to, chemical properties, such as the atom type and the hybridization degree of the atom. The first feature matrix is formed according to the attribute data of each atom, and each row of the first feature matrix represents the attribute data of an atom of the substance to be tested. By performing graph convolution processing on the first adjacent matrix and the first feature matrix, the substance features of the substance to be tested are extracted.

The graph convolution processing of the first adjacent matrix and the first feature matrix are implemented by the following equation (1-1) and equation (1-2).

$$H = {\tilde{D}}^{- \frac{1}{2}} \tilde{A} {\tilde{D}}^{- \frac{1}{2}} X Θ \qquad Equation ( 1 - 1 )$$

$${H}^{( 1 + 1 )} = σ ( {\tilde{D}}^{- \frac{1}{2}} \tilde{A} {\tilde{D}}^{- \frac{1}{2}} {H}^{( 1 )} {Θ}^{( 1 )} ) \qquad Equation ( 1 - 2 )$$

Herein, \tilde{D} represents a degree matrix of A, H represents a convolution result of the first layer graph convolution, \tilde{D} represents a normalized degree matrix D, and the diagonal line of the degree matrix D represents the number of the neighboring atoms of each atom (a neighboring atom of an atom is an atom that has a bond connection with this atom), Ã represents the normalized first adjacent matrix, X represents the first feature matrix, and Θ represents a filter parameter of the first layer graph convolution. H^{(l+1)} represents a convolution result of the (l+1)th layer graph convolution, H^{(l)} represents a convolution result of the lth layer graph convolution, and Θ^{(l)} represents a filter parameter of the lth layer graph convolution, σ( ) represents a nonlinear activation function.

In this way, the first adjacent matrix and the first feature matrix are used to represent the structure features of the substance to be tested, and the substance features of the substance to be tested are extracted by performing graph convolution processing on the first adjacent matrix and the first feature matrix.

In a possible implementation, the substance features of the substance to be tested are obtained according to the first adjacent matrix and the first feature matrix, which includes that: a complementary matrix of the first adjacent matrix is constructed according to a preset input dimension and a dimension of the first adjacent matrix, and a complementary matrix of the first feature matrix is constructed according to the preset input dimension and a dimension of the first feature matrix; the first adjacent matrix and the complementary matrix of the first adjacent matrix are concatenated to obtain a second adjacent matrix with the preset input dimension, and the first feature matrix and the complementary matrix of the first feature matrix are concatenated to obtain a second feature matrix with the preset input dimension; and graph convolution processing is performed on the second adjacent matrix and the second feature matrix to obtain the substance features of the substance to be tested.

For example, the preset input dimension is a preset dimensionality of input data. For example, the preset input dimension is set as 100. After the first adjacent matrix is obtained, it is necessary to determine the dimension of the complementary matrix of the first adjacent matrix according to the dimension of the first adjacent matrix, and then construct the complementary matrix, with the dimension of the complementary matrix, of the first adjacent matrix. For example, it is determined that the difference between the preset input dimension and the dimension of the first adjacent matrix is the dimension of the complementary matrix of the first adjacent matrix. For example, when the preset input dimension is set as 100, the dimension of the first adjacent matrix is 20*20, and the dimension of the first feature matrix is 20*75, it is determined that the dimension of the complementary matrix of the first adjacent matrix is 80*80, and the dimension of the complementary matrix of the first feature matrix is 80*25.

The complementary matrix of the first adjacent matrix is set as a zero matrix or randomly sampled as an adjacent matrix with any neighbor relationships. After obtaining the first feature matrix, it is necessary to determine the dimension of the complementary matrix of the first feature matrix according to the dimension of the first feature matrix, and then construct the complementary matrix, with the dimension of the complementary matrix, of the first feature matrix. For example, it is determined that the difference between the preset input dimension and the dimension of the first feature matrix is the dimension of the complementary matrix of the first feature matrix, the common atoms in the first feature matrix are randomly selected, and the complementary matrix of the first feature matrix is constructed based on the selected atoms.

After the complementary matrix of the first adjacent matrix is constructed, the first adjacent matrix and the complementary matrix of the first adjacent matrix are concatenated to obtain the second adjacent matrix, the dimension of the second adjacent matrix is the preset input dimension*the preset input dimension. After the complementary matrix of the first feature matrix is constructed, the first feature matrix and the complementary matrix of the first feature matrix are concatenated to obtain the second feature matrix, and the dimension of the second feature matrix is the preset input dimension*the dimension of the atom feature. Exemplarily, when the preset input dimension is set as 100 and the dimension of the atom features is 75, it is determined that the dimension of the second adjacent matrix is 100*100, and the dimension of the second feature matrix is 100*75.

The graph convolution processing of the second adjacent matrix and the second feature matrix are implemented by the following equation (1-3), equation (1-4) and equation (1-5).

$${H}^{( 1 , α )} = σ ( ( {( \tilde{D} + {D}^{B} )}^{- \frac{1}{2}} \tilde{A} {( \tilde{D} + {D}^{B} )}^{- \frac{1}{2}} {X ( \tilde{D} + {D}^{B} )}^{- \frac{1}{2}} {B ( {\tilde{D}}^{C} + {D}^{{B}^{T}} )}^{- \frac{1}{2}} {X}^{C} ) Θ ) \qquad Equation ( 1 - 3 )$$

$${H}^{( 1 , β )} = σ ( ( {( {\tilde{D}}^{C} + {D}^{{B}^{T}} )}^{- \frac{1}{2}} {{B}^{T} ( \tilde{D} + {D}^{B} )}^{- \frac{1}{2}} X + {( {\tilde{D}}^{C} + {D}^{{B}^{T}} )}^{- \frac{1}{2}} {{\tilde{A}}^{C} ( {\tilde{D}}^{C} + {D}^{{B}^{T}} )}^{- \frac{1}{2}} {X}^{C} ) Θ ) \qquad Equation ( 1 - 4 )$$

$${H}^{( 1 + 1 )} = [ \begin{matrix}σ ( {\tilde{D}}^{- \frac{1}{2}} \tilde{A} {\tilde{D}}^{- \frac{1}{2}} {H}^{( 1 , α )} {Θ}^{( 1 )} ) \\ σ ( {\tilde{D}}^{{C}^{- \frac{1}{2}}} \tilde{A} {C}^{} {\tilde{D}}^{- \frac{1}{2}} {H}^{( 1 , β )} {Θ}^{( 1 )} )\end{matrix} ] \qquad Equation ( 1 - 5 )$$

Herein, \tilde{D} represents a degree matrix of Ã, [symbol] represents a degree matrix of [symbol] , H^{(1,α)} represents the first n (the number of atoms of the substance to be tested) rows in a convolution result of the first layer, H^{(1,β)} represents the rows in the convolution result of the first layer except for the H^{(1,α)} represents a first conjunction matrix, D^{B} and D^{B}^{^{T}} represent two degree matrices for the rows and columns of the first conjunction matrix B, X represents the first feature matrix, X^{C} represents the complementary matrix of the first feature matrix, Ã^{C} represents the complementary matrix of the normalized first adjacent matrix, \tilde{D}^{C} represents a degree matrix of the complementary matrix of the normalized first adjacent matrix, σ( ) represents a nonlinear activation function, Θ represents a filter parameter of the first layer graph convolution, Θ^{(l)} represents a filter parameter of the lth layer graph convolution. When the first conjunction matrix is zero, that is, the first adjacent matrix has no adjacent relationship with the complementary matrix of the first adjacent matrix, the equations (1-3) and (1-4) are simplified to obtain the equation (1-5).

In this way, the prediction method provided by the embodiments of the present disclosure is suitable for response tests for substances with any size and structure and diseased cells with the target category, and has a strong expansion capability.

In a possible implementation, in the second adjacent matrix, the first adjacent matrix has no adjacent relationship with the complementary matrix of the first adjacent matrix. Here, there is no adjacent relationship between the matrices, which means that the atoms contained in one matrix do not have any connection relationship with the atoms contained in the other matrix.

In the second adjacent matrix obtained by concatenating the first adjacent matrix and the complementary matrix of the first adjacent matrix, the first adjacent matrix has no adjacent relationship with the complementary matrix of the first adjacent matrix. That is to say, the atoms in the substance to be tested and the atoms in the complementary matrix do not have any connection relationship, so that the complementary matrix of the first adjacent matrix constructs the second adjacent matrix whose dimension is the preset input dimension with the first adjacent matrix, and the complementary matrix of the first feature matrix constructs the second adjacent matrix whose dimension is the preset input dimension with the first feature matrix. Because the atoms in the substance to be tested do not have any adjacent relationship with the atoms in the complementary matrix, it will not affect the molecular structure of the substance to be tested, and thus will not affect the test result of the substance to be tested.

In a possible implementation, the first adjacent matrix and the complementary matrix of the first adjacent matrix are concatenated to obtain the second adjacent matrix with the preset input dimension, and the first feature matrix and the complementary matrix of the first feature matrix are concatenated to obtain the second feature matrix with the preset input dimension, which include that: a first conjunction matrix is constructed according to the first adjacent matrix and the complementary matrix of the first adjacent matrix, herein, elements in the first conjunction matrix are all preset values; the first adjacent matrix and the complementary matrix of the first adjacent matrix are connected through the first conjunction matrix to obtain the second adjacent matrix with the preset input dimension; and the first feature matrix and the complementary matrix of the first feature matrix are connected to obtain the second feature matrix with the preset input dimension.

For example, the first conjunction matrix whose elements are all 0 is constructed. The first conjunction matrix, the first adjacent matrix, and the complementary matrix of the first adjacent matrix form the second adjacent matrix. In the second adjacent matrix, the first conjunction matrix connects the first adjacent matrix and the complementary matrix of the first adjacent matrix, so that the first adjacent matrix has no adjacent relationship with the complementary matrix of the first adjacent matrix. Exemplarily, FIG. 2 is a diagram of matrices provided by an embodiment of the present disclosure. As shown in FIG. 2, in the second adjacent matrix with a dimension of 100*100, the first adjacent matrix with a dimension of 20*20 is located at an upper left position of the second adjacent matrix, the complementary matrix, with a dimension of 80*80, of the first adjacent matrix is located at a lower right position of the second adjacent matrix, the first conjunction matrix with a dimension of 20*80 is located below the first adjacent matrix and at a left side of the complementary matrix of the first adjacent matrix, and the first conjunction matrix with a dimension of 80*20 is located at a right side of the first adjacent matrix and above the complementary matrix of the first adjacent matrix.

It should be noted that the FIG. 2 illustrates only an example of a first conjunction matrix connecting the first adjacent matrix and the complementary matrix of the first adjacent matrix. In fact, any connection method that makes the first adjacent matrix have no adjacent relationship with the complementary matrix of the first adjacent matrix is adopted. For example, the first adjacent matrix with the dimension of 20*20 is located at the lower right position of the second adjacent matrix, and the complementary matrix, with the dimension of 80*80, of the first adjacent matrix is located at the upper left position of the second adjacent matrix, the first conjunction matrix with the dimension of 80*20 is located above the first adjacent matrix and at the right side of the complementary matrix of the first adjacent matrix, and the first conjunction matrix with the dimension of 20*80 is located at the left side of the first adjacent matrix and below the complementary matrix of the first adjacent matrix. The present disclosure does not specifically limit the manner in which the first conjunction matrix connects the first adjacent matrix and the complementary matrix of the first adjacent matrix.

Correspondingly, a connection method between the first feature matrix and the complementary matrix of the first feature matrix is determined according to a connection method between the first adjacent matrix and the complementary matrix of the first adjacent matrix. For example, referring to the connection method between the first adjacent matrix and the complementary matrix of the first adjacent matrix shown in FIG. 2, the connection method of the first feature matrix and the complementary matrix of the first feature matrix is that the first feature matrix is located at the upper position and the complementary matrix of the first feature matrix is located at the lower position.

It should be noted that in a case that the connection method between the first adjacent matrix and the complementary matrix of the first adjacent matrix is that the first adjacent matrix is located at the lower right position of the second adjacent matrix and the complementary matrix of the first adjacent matrix is located at the upper left position of the second adjacent matrix, in the second feature matrix, the first feature matrix is located at the lower position and the complementary matrix of the first feature matrix is located at the upper position.

In this way, the substance features of the substance to be tested are constructed as input data that meets the requirements of the response test, and the molecular structure of the substance to be tested will not be affected, and thus the result of the response test for the substance to be tested will not be affected.

In a possible implementation, the cell feature extraction is performed on the diseased cell of the target category to obtain the at least one cell feature of the diseased cell, which includes at least one of the following.

Feature extraction is performed on genomic mutation of the diseased cell to obtain a genome feature of the diseased cell; feature extraction is performed on gene expression of the diseased cell to obtain a transcriptome feature of the diseased cell; or feature extraction is performed on deoxyribonucleic acid (DNA) methylation data of the diseased cell to obtain an epigenome feature of the diseased cell.

For example, after the diseased cell of the target category is determined, the genomic mutation, gene expression and DNA methylation data of the diseased cell are acquired. The acquisition process is completed by performing extraction by adopting the related arts, or performing query directly from the database, which will not be repeated in the present disclosure.

Exemplarily, the genomic mutation, gene expression, and DNA methylation data of the diseased cell are preprocessed into fixed-dimensional vectors in advance. For example, the genomic mutation of the diseased cell is preprocessed into a 34673-dimensional vector, and the gene expression of the diseased cell is preprocessed into a 697-dimensional vector, and the DNA methylation data of the diseased cell is preprocessed into an 808-dimensional vector. The convolutional neural network for extracting the genome feature is pre-trained and is used to perform feature extraction on the preprocessed genomic mutation of the diseased cell to obtain the genome feature of the diseased cell; the convolutional neural network for extracting the transcriptome feature is pre-trained and is used to perform feature extraction on the preprocessed gene expression of the diseased cell to obtain the transcriptome feature of the diseased cell; and the convolutional neural network for extracting the epigenome feature is pre-trained and is used to perform feature extraction on the preprocessed DNA methylation data to obtain the epigenome feature of diseased cell. Herein, the dimension of the genome feature, the dimension of the transcriptome feature, and the dimension of the epigenome feature are identical to the dimension of substance feature. In a possible implementation, the convolutional neural network for extracting the cell feature is a multi-modal sub-neural network.

In a possible implementation, the cell feature include the genome feature, the transcriptome feature, and the epigenome feature; and the substance features and the at least one cell feature are concatenated to obtain the combined feature after concatenation, which includes that: the substance features and at least one of the genome feature, the transcriptome feature or the epigenome feature are concatenated to obtain the combined feature after concatenation.

Exemplarily, the combined feature is obtained by concatenating the substance features of the substance to be tested with the genome feature, the transcriptome feature, and the epigenome feature. The combined feature is represented as: substance feature+genome feature+transcriptome feature+epigenome feature. The convolution processing is performed on the combined feature to obtain the response result of the substance to be tested against the diseased cell.

In this way, multiple cell features of the diseased cell are learned in a multi-modal manner, and the response result is predicted based on sufficient cell features, which will improve the accuracy of the predicted result.

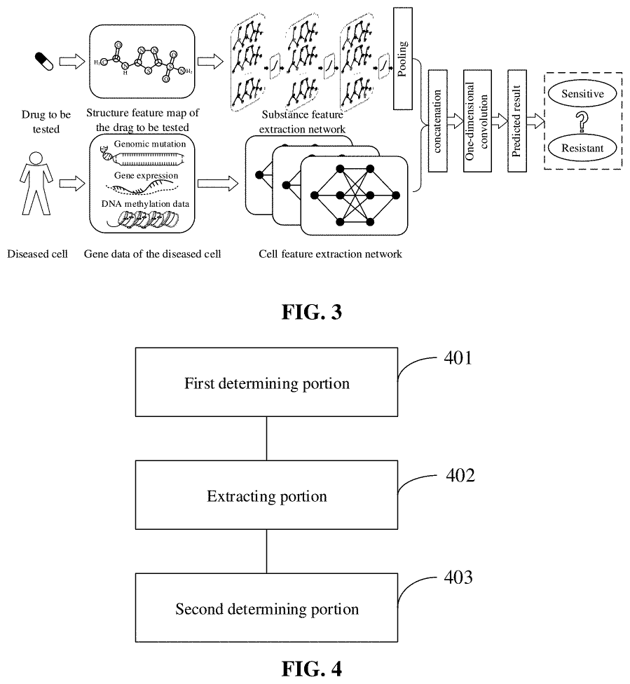

In order to enable those skilled in the art to better understand the embodiments of the present disclosure, the embodiments of the present disclosure are described below through the example shown in FIG. 3.

FIG. 3 is a flowchart of the prediction method provided by an embodiment of the present disclosure. As shown in FIG. 3, the substance to be tested is a drug and the diseased cell is a cancer cell. A structure feature map of the drug to be tested is constructed according to the molecular structure of the drug to be tested, and feature extraction is performed on the structure feature map through a substance feature extraction network to obtain the substance features of the drug to be tested. Genomic mutation, gene expression and DNA methylation data of the cancer cell are obtained, and cell feature extraction is performed through a cell feature extraction network. The cell feature extraction network includes: a genome feature extraction network, a transcriptome feature extraction network, and an epigenome feature extraction network. The feature extraction is performed on the genomic mutation through the genome feature extraction network to obtain genome feature(s) of the cancer cell, the feature extraction is performed on the gene expression through the transcriptome feature extraction network to obtain transcriptome feature(s) of the cancer cell, and the feature extraction is performed on the DNA methylation data through the epigenome feature extraction network to obtain epigenome feature(s) of the cancer cell. After pooling processing is performed on the substance features of the drug to be tested, the pooled substance features are concatenated with the genome feature(s), the transcriptome feature(s) and the epigenome feature(s) to obtain a combined feature, and convolution processing is performed on the combined feature to obtain a predicted response result of the drug to be tested against the cancer cell, herein, the response result indicates whether the drug to be tested is sensitive or resistant to the cancer cell.

In a possible implementation, the method is implemented by a neural network, and the method further includes: the neural network is trained based on a preset training set, herein, the training set includes multiple groups of sample data, and each group of sample data includes a structure feature map of a sample substance, genomic mutation of a sample diseased cell, gene expression of the sample diseased cell, DNA methylation data of the sample diseased cell, and a labeled response result of the sample substance against the sample diseased cell.

In a possible implementation, the neural network is a uniform graph convolutional neural network.

In a possible implementation, the neural network includes a first feature extraction network, a second feature extraction network and a prediction network; and the neural network is trained based on the preset training set, which includes that: feature extraction is performed on the structure feature map of the sample substance through the first feature extraction network to obtain sample substance features of the sample substance; a sample genome feature corresponding to the genomic mutation of the sample diseased cell, a sample transcriptome feature corresponding to the gene expression of the sample diseased cell, and a sample epigenome feature corresponding to the DNA methylation data of the sample diseased cell are respectively extracted through the second feature extraction network; convolution processing is performed, through the prediction network, on a combined sample feature obtained after concatenation of the sample substance features, the sample genome feature, the sample transcriptome feature and the sample epigenome feature, to predict a response result of the sample substance against the sample diseased cell; a predicted loss of the neural network is determined according to the predicted response result and the labeled response result; and the neural network is trained according to the predicted loss.

For example, the feature extraction is performed on the structure feature map of the sample substance through the first feature extraction network to obtain the sample substance features of the sample substance. The second feature extraction network includes a first sub-network, a second sub-network, and a third sub-network. The feature extraction is performed on genomic mutation of the sample diseased cell through the first sub-network to obtain the sample genome feature(s). The feature extraction is performed on gene expression of the sample diseased cell through the second sub-network to obtain the sample transcriptome feature(s). The feature extraction is performed on DNA methylation data of the sample diseased cell through the third sub-network to obtain the sample epigenome feature(s). The sample substance features, the sample genome feature(s), the sample transcriptome feature(s), and the sample epigenome feature(s) are concatenated to obtain the combined sample feature. The convolution processing is performed on the combined sample feature through the prediction network to obtain the response result of the sample substance to the sample diseased cell. The predicted loss of the neural network is determined according to the response result and the labeled response result, and the network parameter of the neural network is adjusted according to the predicted loss to make the predicted loss of the neural network meet the training requirements, for example, make the predicted loss of the neural network less than a training threshold.

It should be understood that without violating the principle and logic, the various method embodiments provided in the embodiments of the present disclosure are combined with each other to form a combined embodiment, which will not be repeated in this disclosure due to space constraints. Those skilled in the art can understand that in the above-mentioned methods of the specific implementation, the specific execution order of each operation should be determined by its function and possible internal logic.

In addition, the embodiments of the present disclosure also provide a prediction device, an electronic device, a computer-readable storage media and programs, all of which are used to implement any kind of prediction method provided by the embodiments of the present disclosure. The corresponding technical solutions and descriptions refer to corresponding records of the method embodiments, which will not be repeated herein.

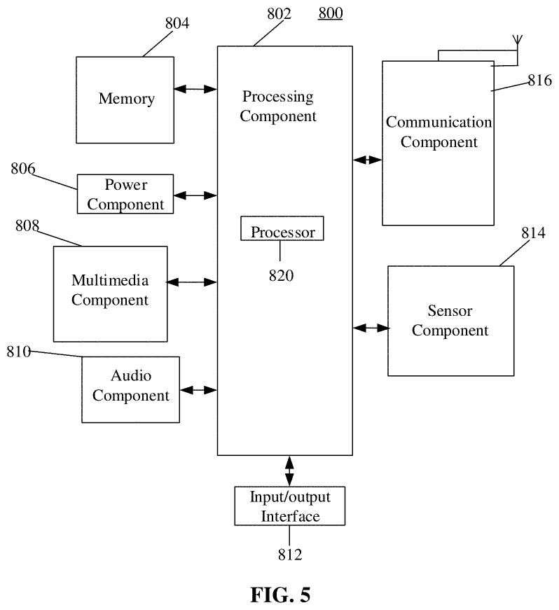

FIG. 4 is a structural diagram of a prediction device provided by an embodiment of the present disclosure. As shown in FIG. 4, the prediction device includes a first determining portion 401, an extracting portion 402 and a second determining portion 403.

The first determining portion 401 is configured to: according to a molecular structure of a substance to be tested, determine substance features of the substance to be tested.

The extracting portion 402 is configured to extract at least one cell feature of a diseased cell of a target category to obtain the at least one cell feature of the diseased cell.

The second determining portion 403 is configured to: according to the substance features and the at least one cell feature, predict a response result of the substance to be tested against the diseased cell.

In this way, according to a molecular structure of a substance to be tested, a structure feature map of the substance to be tested is constructed; based on the structure feature map, the substance features of the substance to be tested are extracted; at least one cell feature of a diseased cell of a target category is extracted; and the response result of the substance to be tested against the diseased cell is predicted according to the substance features of the substance to be tested and the at least one cell feature of the diseased cell. According to the prediction device provided by the embodiment of the present disclosure, the substance features of the substance to be tested are extracted based on the structure feature map of the substance to be tested, and the substance features extracted in this way are denser than the substance features extracted manually, thereby improving the accuracy of the test result and the efficiency of obtaining the test result.

In a possible implementation, the first determining portion 401 is configured to: according to the molecular structure of the substance to be tested, construct a structure feature map of the substance to be tested, herein, the structure feature map includes at least two nodes and lines between the nodes, each node represents an atom in the molecular structure, and each line represents an atomic bond in the molecular structure; and according to the structure feature map, determine the substance features of the substance to be tested.

In a possible implementation, the first determining portion 401 is further configured to: according to the structure feature map, obtain a first adjacent matrix and a first feature matrix of the substance to be tested, herein, the first adjacent matrix represents neighbor relationships between atoms of the substance to be tested, and the first feature matrix represents attribute data of each atom of the substance to be tested; and according to the first adjacent matrix and the first feature matrix, obtain the substance features of the substance to be tested.

In a possible implementation, the first determining portion 401 is further configured to: according to a preset input dimension and a dimension of the first adjacent matrix, construct a complementary matrix of the first adjacent matrix, and according to the preset input dimension and a dimension of the first feature matrix, construct a complementary matrix of the first feature matrix; concatenate the first adjacent matrix and the complementary matrix of the first adjacent matrix to obtain a second adjacent matrix with the preset input dimension, and concatenate the first feature matrix and the complementary matrix of the first feature matrix to obtain a second feature matrix with the preset input dimension; and perform graph convolution processing on the second adjacent matrix and the second feature matrix to obtain the substance features of the substance to be tested.

In a possible implementation, in the second adjacent matrix, the first adjacent matrix has no adjacent relationship with the complementary matrix of the first adjacent matrix.

In a possible implementation, the first determining portion 401 is further configured to: according to the first adjacent matrix and the complementary matrix of the first adjacent matrix, construct a first conjunction matrix; connect the first adjacent matrix and the complementary matrix of the first adjacent matrix via the first conjunction matrix to obtain the second adjacent matrix with the preset input dimension; and connect the first feature matrix and the complementary matrix of the first feature matrix to obtain the second feature matrix with the preset input dimension.

In a possible implementation, the extracting portion 402 is configured to perform at least one of: performing feature extraction on genomic mutation of the diseased cell to obtain a genome feature of the diseased cell; performing feature extraction on gene expression of the diseased cell to obtain a transcriptome feature of the diseased cell; or, performing feature extraction on DNA methylation data of the diseased cell to obtain an epigenome feature of the diseased cell.

In a possible implementation, the second determining portion 403 is configured to: concatenate the substance features and the at least one cell feature to obtain a combined feature after concatenation; and perform convolution processing on the combined feature to obtain the response result of the substance to be tested against the diseased cell.

In a possible implementation, the cell feature includes the genome feature, the transcriptome feature, and the epigenome feature, and the second determining portion 403 is further configured to: concatenate the substance features and at least one of the genome feature, the transcriptome feature, or the epigenome feature to obtain the combined feature after concatenation.

In a possible implementation, the device is implemented by a neural network, and the device further includes: a training portion, configured to train the neural network based on a preset training set, herein, the training set includes multiple groups of sample data, and each group of sample data includes a structure feature map of a sample substance, genomic mutation of a sample diseased cell, gene expression of the sample diseased cell, DNA methylation data of the sample diseased cell, and a labeled response result of the sample substance against the sample diseased cell.

In a possible implementation, the neural network includes a first feature extraction network, a second feature extraction network, and a prediction network; and the training portion is further configured to: perform feature extraction on the structure feature map of the sample substance via the first feature extraction network to obtain sample substance features of the sample substance; extract the sample genome feature corresponding to the genomic mutation of the sample diseased cell, the sample transcriptome feature corresponding to the gene expression of the sample diseased cell, and the sample epigenome feature corresponding to the DNA methylation data of the sample diseased cell respectively via the second feature extraction network; perform convolution processing, via the prediction network, on a combined sample feature obtained after concatenation of the sample substance feature, the sample genome feature, the sample transcriptome feature and the sample epigenome feature to obtain a response result of the sample substance against the sample diseased cell; according to the response result and the labeled response result, determine the predicted loss of the neural network; and according to the predicted loss, train the neural network.

In some embodiments, the functions owned by, or parts contained in the device provided by the embodiments of the present disclosure are configured to perform the methods described in the above method embodiments. The specific implementation refers to the description of the above method embodiments, which will not be repeated herein.

In the embodiments of the present disclosure and other embodiments, “portion” is a part of circuits, a part of processors, a part of programs or software, etc. Of course, the “portion” are also units, modules, or non-modular.

The embodiment of the present disclosure also provides a computer-readable storage medium, having stored thereon computer program instructions that, when executed by a processor, implement the above-mentioned method. The computer-readable storage medium is a non-transitory computer-readable storage medium.

The embodiment of the present disclosure also provides an electronic device, including: a processor; a memory configured to store instructions executable by the processor; herein, the processor is configured to invoke instructions stored in the memory to perform the above method.

The embodiment of the present disclosure also provides a computer program product including computer-readable codes. When the computer-readable codes are run on a device, a processor in the device executes instructions configured to implement the prediction method provided by any of the above embodiments.

The embodiment of the present disclosure also provides another computer program product configured to store computer-readable instructions that cause the computer to perform the operations of the prediction method provided in any of the foregoing embodiments when the instructions are executed.

The electronic device is provided as a terminal, a server or other form of device.

FIG. 5 is a structural diagram of an electronic device provided by an embodiment of the present disclosure. For example, the electronic device 800 is a terminal, such as a mobile phone, a computer, a digital broadcasting terminal, a message transceiver, a game console, a tablet device, a medical device, a fitness device, a personal digital assistant or the like.

Referring to FIG. 5, the electronic device 800 includes one or more of the following components: a processing component 802, a memory 804, a power supply component 806, a multimedia component 808, an audio component 810, an input/output (I/O) interface 812, a sensor component 814 and a communication component 816.

The processing component 802 generally controls the overall operations of the electronic device 800, such as operations associated with display, telephone calls, data communications, camera operations, and recording operations. The processing component 802 includes one or more processors 820 to execute instructions to complete all or part of the operations of the foregoing method. In addition, the processing component 802 includes one or more modules to facilitate the interaction between the processing component 802 and other components. For example, the processing component 802 includes a multimedia module to facilitate the interaction between the multimedia component 808 and the processing component 802.

The memory 804 is configured to store various types of data to support operations in the electronic device 800. Examples of these data include instructions for any application or method operating on the electronic device 800, contact data, phone book data, messages, pictures, videos, etc. The memory 804 is implemented by any type of volatile or non-volatile storage device or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk or an optical disk.

The power supply component 806 provides power for various components of the electronic device 800. The power supply component 806 includes a power management system, one or more power supplies, and other components associated with the generation, management, and distribution of power for the electronic device 800.

The multimedia component 808 includes a screen that provides an output interface between the electronic device 800 and the user. In some embodiments, the screen includes a liquid crystal display (LCD) and a touch panel (TP). If the screen includes a touch panel, the screen is implemented as a touch screen to receive input signals from the user. The touch panel includes one or more touch sensors to sense touch, sliding, and gestures on the touch panel. The touch sensor not only senses the boundary of a touch or slide action, but also detects the duration and pressure related to the touch or slide operation. In some embodiments, the multimedia component 808 includes a front camera and/or a rear camera.

When the electronic device 800 is in an operation mode, such as a shooting mode or a video mode, the front camera and/or the rear camera will receive external multimedia data. Each front camera and rear camera is a fixed optical lens system or have focal length and optical zoom capabilities.

The audio component 810 is configured to output and/or input audio signals. For example, the audio component 810 includes a microphone (MIC). When the electronic device 800 is in an operation mode, such as a calling mode, a recording mode, and a voice recognition mode, the microphone is configured to receive external audio signals. The received audio signals are further stored in the memory 804 or transmitted via the communication component 816. In some embodiments, the audio component 810 further includes a speaker for outputting audio signals.

The I/O interface 812 provides an interface between the processing component 802 and a peripheral interface module. The peripheral interface module is a keyboard, a click wheel, a button, and the like. These buttons include but are not limited to a home button, a volume button, a start button and a lock button.

The sensor component 814 includes one or more sensors for providing the electronic device 800 with various aspects of state evaluation. For example, the sensor component 814 detects the on/off status of the electronic device 800 and the relative positioning of the components, the components are the display and the keypad of the electronic device 800. The sensor component 814 also detect the position change of the electronic device 800 or a component of the electronic device 800, the presence or absence of contact between the user and the electronic device 800, the orientation, acceleration or deceleration of the electronic device 800, and the temperature change of the electronic device 800. The sensor component 814 includes a proximity sensor configured to detect the presence of nearby objects when there is no physical contact. The sensor component 814 also includes a light sensor, such as a CMOS or a CCD image sensor, for being used in imaging applications. In some embodiments, the sensor component 814 also includes an acceleration sensor, a gyroscope sensor, a magnetic sensor, a pressure sensor or a temperature sensor.

The communication component 816 is configured to facilitate wired or wireless communication between the electronic device 800 and other devices. The electronic device 800 accesses a wireless network based on a communication standard, such as WiFi, 2G, or 3G, or a combination thereof. In an exemplary embodiment, the communication component 816 receives a broadcast signal or broadcast related information from an external broadcast management system via broadcast channel. In an exemplary embodiment, the communication component 816 further includes a near field communication (NFC) module to facilitate short-range communication. For example, the NFC module is implemented based on radio frequency identification (RFID) technologies, infrared data association (IrDA) technologies, ultra-wideband (UWB) technologies, Bluetooth (BT) technologies and other technologies.

In an exemplary embodiment, the electronic device 800 is implemented by one or more application specific integrated circuits (ASIC), digital signal processors (DSP), digital signal processing devices (DSPD), programmable logic devices (PLD), field programmable gate arrays (FPGA), controllers, microcontrollers, microprocessors, or other electronic component implementations configured to perform the above methods.

In an exemplary embodiment, there is also provided a non-transitory computer-readable storage medium, such as the memory 804 including computer program instructions that are executed by the processor 820 of the electronic device 800 to complete the foregoing method.

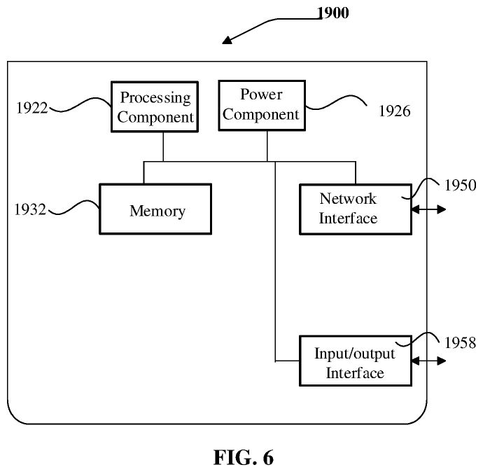

FIG. 6 is a structural diagram of an electronic device provided by an embodiment of the present disclosure. For example, the electronic device 1900 is provided as a server. Referring to FIG. 6, the electronic device 1900 includes a processing component 1922, which further includes one or more processors, and a memory resource represented by the memory 1932 configured to store instructions executable by the processing component 1922, such as application programs. The application program stored in the memory 1932 includes one or more parts each of which corresponds to a set of instructions. In addition, the processing component 1922 is configured to execute instructions to perform the prediction method.

The electronic device 1900 also includes a power supply component 1926 configured to perform power management of the electronic device 1900, a wired or wireless network interface 1950 configured to connect the electronic device 1900 to a network, and an input/output (I/O) interface 1958. The electronic device 1900 operates an operating system stored in the memory 1932, such as Windows Server™, Mac OS X™, Unix™, Linux™, FreeBSD™ or the like.

In an exemplary embodiment, there is also provided a non-transitory computer-readable storage medium, such as the memory 1932 including computer program instructions which are executed by the processing component 1922 of the electronic device 1900 to complete the foregoing method.

The present disclosure is a system, a method, and/or a computer program product. The computer program product includes a computer-readable storage medium loaded with computer-readable program instructions that cause a processor to implement various aspects of the present disclosure.

The computer-readable storage medium is a tangible device that holds and stores instructions used by the instruction execution device. The computer-readable storage medium is, for example, but not limited to, an electrical storage device, a magnetic storage device, an optical storage device, an electromagnetic storage device, a semiconductor storage device, or any suitable combination of the foregoing. More specific examples (non-exhaustive list) of computer readable storage media include: a portable computer disk, a hard disk, a random access memory (RAM), a read only memory (ROM), an erasable programmable read only memory (EPROM or a flash memory), a static random access memory (SRAM), a portable compact disk read-only memory (CD-ROM), a digital versatile disk (DVD), a memory stick, a floppy disk, a mechanical encoding device, such as a punch card or a protruding structure in the groove having stored thereon instructions, and any suitable combination of the above. The computer-readable storage medium used here is not interpreted as a transient signal itself, such as radio waves or other freely propagating electromagnetic waves, electromagnetic waves transmitting through waveguides or other transmission media (for example, light pulses transmitting through fiber optic cables), or electrical signals transmitting through electric wires.

The computer-readable program instructions described herein are downloaded from a computer-readable storage medium to various computing/processing devices, or downloaded to an external computer or external storage device via network, such as the Internet, a local area network, a wide area network, and/or a wireless network. The network includes copper transmission cables, optical fiber transmission, wireless transmission, routers, firewalls, switches, gateway computers, and/or edge servers. The network adapter card or network interface in each computing/processing device receives computer-readable program instructions from the network, and forwards the computer-readable program instructions for storage in the computer-readable storage medium in each computing/processing device.

The computer program instructions used to perform the operations of the present disclosure are assembly instructions, instruction set architecture (ISA) instructions, machine instructions, machine-related instructions, microcode, firmware instructions, status setting data, or source codes or object codes written by any combination of one or more programming languages, the programming language includes object-oriented programming languages such as Smalltalk, C++, etc., and conventional procedural programming languages such as “C” language or similar programming languages. Computer-readable program instructions are executed entirely on the computer of the user, partly on the computer of the user, executed as a stand-alone software package, partly on the computer of the user and partly on a remote computer, or entirely on the remote computer or a server. In the case related to the remote computer, the remote computer is connected to the computer of the user through any kind of network, including a local area network (LAN) or a wide area network (WAN), or the remote computer can be connected to an external computer (for example, using an Internet service provider to provide an Internet connection). In some embodiments, an electronic circuit, such as a programmable logic circuit, a field programmable gate array (FPGA), or a programmable logic array (PLA), is customized by using the status information of the computer-readable program instructions. The computer-readable program instructions are executed to realize various aspects of the present disclosure.

Herein, various aspects of the present disclosure are described with reference to flowcharts and/or block diagrams of methods, devices (systems) and computer program products according to embodiments of the present disclosure. It should be understood that each block of the flowchart and/or block diagram and the combination of each block in the flowchart and/or each block in block diagram are implemented by computer readable program instructions.

These computer-readable program instructions are provided to a processor of a general-purpose computer, a special-purpose computer, or other programmable data processing device, thereby producing a machine that makes these instructions, when executed by the processor of the computer or other programmable data processing device, produce a device that implements the functions/actions specified in one or more blocks in the flowchart and/or block diagram. It is also possible to store these computer-readable program instructions in a computer-readable storage medium. These instructions make computers, programmable data processing devices, and/or other devices work in a specific manner, so that the computer-readable medium storing instructions includes a manufacture, which includes instructions for implementing various aspects of the functions/actions specified in one or more blocks in the flowchart and/or the block diagram.

It is also possible to load computer-readable program instructions on a computer, other programmable data processing devices, or other equipment, so that a series of operations are executed on the computer, other programmable data processing device, or other equipment to produce a computer-implemented process, so that the instructions executed on the computer, other programmable data processing device, or other equipment implement the functions/actions specified in one or more blocks in the flowcharts and/or block diagrams.

The flowcharts and block diagrams in the accompanying drawings show the architecture, functions, and operations that are possibly implemented by the system, method, and computer program product according to multiple embodiments of the present disclosure. In this regard, each block in the flowchart or block diagram represents a module, a program segment, or a part of an instruction, and the module, the program segment, or the part of an instruction contains one or more executable instructions for implementing the specified logical functions. In some alternative implementations, the functions marked in the block also occur in an order different from the order marked in the drawings. For example, two consecutive blocks actually are executed substantially in parallel, or these two consecutive blocks sometimes are executed in the reverse order, which depends on the functions involved. It should also be noted that each block in the block diagram and/or flowchart, and the combination of the blocks in the block diagram and/or flowchart, are implemented by a dedicated hardware-based system that performs the specified functions or actions or they are implemented by a combination of dedicated hardware and computer instructions.

The computer program product is specifically implemented by hardware, software or a combination thereof. In an optional embodiment, the computer program product is specifically embodied as a computer storage medium. In another optional embodiment, the computer program product is specifically embodied as a software product, such as a software development kit (SDK), etc.

The embodiments of the present disclosure have been described above, the above description is exemplary, not exhaustive, and is not limited to the disclosed embodiments. Without departing from the scope and spirit of the illustrated embodiments, many modifications and changes are obvious to those of ordinary skilled in the art. The choice of terms used herein is intended to best explain the principles, practical applications, or improvements to the technology in the market for each embodiment, or to enable other ordinary skilled in the art to understand the various embodiments disclosed herein.

### INDUSTRIAL APPLICABILITY

In the embodiments of the present disclosure, substance features of a substance to be tested are determined according to a molecular structure of the substance to be tested, and at least one cell feature of a diseased cell of a target category is extracted; and according to the substance features of the substance to be tested and the at least one cell feature of the diseased cell, a response result of the substance to be tested against the diseased cell is predicted. According to the prediction method and device, the electronic device, and the storage medium provided by the embodiments of the present disclosure, the substance features of the substance to be tested are extracted based on a structure feature map of the substance to be tested, and the substance features extracted in this way are denser than the substance features extracted manually, thereby improving the accuracy of the test result and the efficiency of obtaining the test result.

1. A prediction method, comprising:
according to a molecular structure of a substance to be tested, determining substance features of the substance to be tested;
performing feature extraction on a diseased cell of a target category to obtain at least one cell feature of the diseased cell; and
according to the substance features and the at least one cell feature, predicting a response result of the substance to be tested against the diseased cell.

2. The prediction method of claim 1, wherein determining the substance features of the substance to be tested according to the molecular structure of the substance to be tested comprises:
according to the molecular structure of the substance to be tested, constructing a structure feature map of the substance to be tested, wherein the structure feature map includes at least two nodes and lines between the nodes, each node represents an atom in the molecular structure, and each line represents an atomic bond in the molecular structure; and
according to the structure feature map, determining the substance features of the substance to be tested.

3. The prediction method of claim 2, wherein determining the substance features of the substance to be tested according to the structure feature map comprises:
according to the structure feature map, obtaining a first adjacent matrix and a first feature matrix of the substance to be tested, wherein the first adjacent matrix represents neighbor relationships between atoms of the substance to be tested, and the first feature matrix represents attribute data of each atom of the substance to be tested; and
according to the first adjacent matrix and the first feature matrix, obtaining the substance features of the substance to be tested.

4. The prediction method of claim 3, wherein obtaining the substance features of the substance to be tested according to the first adjacent matrix and the first feature matrix comprises:
constructing a complementary matrix of the first adjacent matrix according to a preset input dimension and a dimension of the first adjacent matrix, and constructing a complementary matrix of the first feature matrix according to the preset input dimension and a dimension of the first feature matrix;
concatenating the first adjacent matrix and the complementary matrix of the first adjacent matrix to obtain a second adjacent matrix with the preset input dimension, and concatenating the first feature matrix and the complementary matrix of the first feature matrix to obtain a second feature matrix with the preset input dimension; and
performing graph convolution processing on the second adjacent matrix and the second feature matrix to obtain the substance features of the substance to be tested.

5. The prediction method of claim 4, wherein, in the second adjacent matrix, the first adjacent matrix has no adjacent relationship with the complementary matrix of the first adjacent matrix.

6. The prediction method of claim 4, wherein concatenating the first adjacent matrix and the complementary matrix of the first adjacent matrix to obtain the second adjacent matrix with the preset input dimension, and concatenating the first feature matrix and the complementary matrix of the first feature matrix to obtain the second feature matrix with the preset input dimension comprises:
according to the first adjacent matrix and the complementary matrix of the first adjacent matrix, constructing a first conjunction matrix, wherein elements in the first conjunction matrix are all preset values;
connecting the first adjacent matrix and the complementary matrix of the first adjacent matrix through the first conjunction matrix to obtain the second adjacent matrix with the preset input dimension; and
connecting the first feature matrix and the complementary matrix of the first feature matrix to obtain the second feature matrix with the preset input dimension.

7. The prediction method of claim 1, wherein performing feature extraction on the diseased cell of the target category to obtain the at least one cell feature of the diseased cell comprises at least one of:
performing feature extraction on genomic mutation of the diseased cell to obtain a genome feature of the diseased cell;
performing feature extraction on gene expression of the diseased cell to obtain a transcriptome feature of the diseased cell; or
performing feature extraction on Deoxyribonucleic Acid (DNA) methylation data of the diseased cell to obtain an epigenome feature of the diseased cell.

8. The prediction method of claim 1, wherein predicting the response result of the substance to be tested against the diseased cell according to the substance features and the at least one cell feature comprises:
concatenating the substance features and the at least one cell feature to obtain a combined feature after concatenation; and
performing convolution processing on the combined feature to obtain a predicted response result of the substance to be tested against the diseased cell.

9. The prediction method of claim 8, wherein the at least one cell feature includes at least one genome feature, at least one transcriptome feature, and at least one epigenome feature, and
wherein concatenating the substance features and the at least one cell feature to obtain the combined feature after concatenation comprises:
concatenating the substance features and at least one of the genome feature, the transcriptome feature or the epigenome feature to obtain the combined feature after concatenation.

10. The prediction method of claim 1, wherein the method is implemented by a neural network and comprises:
training the neural network based on a preset training set, wherein the preset training set comprises a plurality of groups of sample data, and each group of sample data comprises: a structure feature map of a sample substance, genomic mutation of a sample diseased cell, gene expression of the sample diseased cell, Deoxyribonucleic Acid (DNA) methylation data of the sample diseased cell, and a labeled response result of the sample substance against the sample diseased cell.

11. The prediction method of claim 10, wherein the neural network comprises a first feature extraction network, a second feature extraction network and a prediction network; and
wherein training the neural network based on the preset training set comprises:
performing feature extraction on the structure feature map of the sample substance through the first feature extraction network to obtain sample substance features of the sample substance;
extracting, through the second feature extraction network, at least one sample genome feature corresponding to the genomic mutation of the sample diseased cell, at least one sample transcriptome feature corresponding to the gene expression of the sample diseased cell, and at least one sample epigenome feature corresponding to the DNA methylation data of the sample diseased cell;
performing, through the prediction network, convolution processing on a sample combined feature obtained after concatenation of the sample substance features, the sample genome feature, the sample transcriptome feature and the sample epigenome feature, to obtain a response result of the sample substance against the sample diseased cell;
determining a predicted loss of the neural network according to the response result and the labeled response result; and
training the neural network according to the predicted loss.

12. An electronic device, comprising:
a processor; and
a memory, configured to store instructions that, when executed by the processor, cause the processor to perform the following operations including:
according to a molecular structure of a substance to be tested, determining substance features of the substance to be tested;
performing feature extraction on a diseased cell of a target category to obtain at least one cell feature of the diseased cell; and
according to the substance features and the at least one cell feature, predicting a response result of the substance to be tested against the diseased cell.

13. The electronic device of claim 12, wherein the processor is further configured to:
according to the molecular structure of the substance to be tested, construct a structure feature map of the substance to be tested, wherein the structure feature map includes at least two nodes and lines between the nodes, each node represents an atom in the molecular structure, and each line represents an atomic bond in the molecular structure; and
according to the structure feature map, determine the substance features of the substance to be tested.

14. The electronic device of claim 13, wherein the processor is further configured to:
according to the structure feature map, obtain a first adjacent matrix and a first feature matrix of the substance to be tested, wherein the first adjacent matrix represents neighbor relationships between atoms of the substance to be tested, and the first feature matrix represents attribute data of each atom of the substance to be tested; and
according to the first adjacent matrix and the first feature matrix, obtain the substance features of the substance to be tested.

15. The electronic device of claim 14, wherein the processor is further configured to:
construct a complementary matrix of the first adjacent matrix according to a preset input dimension and a dimension of the first adjacent matrix, and construct a complementary matrix of the first feature matrix according to the preset input dimension and a dimension of the first feature matrix;
concatenate the first adjacent matrix and the complementary matrix of the first adjacent matrix to obtain a second adjacent matrix with the preset input dimension, and concatenate the first feature matrix and the complementary matrix of the first feature matrix to obtain a second feature matrix with the preset input dimension; and
perform graph convolution processing on the second adjacent matrix and the second feature matrix to obtain the substance features of the substance to be tested.

16. The electronic device of claim 15, wherein, in the second adjacent matrix, the first adjacent matrix has no adjacent relationship with the complementary matrix of the first adjacent matrix.

17. The electronic device of claim 15, wherein the processor is further configured to:
according to the first adjacent matrix and the complementary matrix of the first adjacent matrix, construct a first conjunction matrix, wherein elements in the first conjunction matrix are all preset values;
connect the first adjacent matrix and the complementary matrix of the first adjacent matrix through the first conjunction matrix to obtain the second adjacent matrix with the preset input dimension; and
connect the first feature matrix and the complementary matrix of the first feature matrix to obtain the second feature matrix with the preset input dimension.

18. The electronic device of claim 12, wherein the processor is further configured to perform at least one of:
performing feature extraction on genomic mutation of the diseased cell to obtain a genome feature of the diseased cell;
performing feature extraction on gene expression of the diseased cell to obtain a transcriptome feature of the diseased cell; or
performing feature extraction on Deoxyribonucleic Acid (DNA) methylation data of the diseased cell to obtain an epigenome feature of the diseased cell.

19. The electronic device of claim 12, wherein the processor is further configured to:
concatenate the substance features and the at least one cell feature to obtain a combined feature after concatenation; and
perform convolution processing on the combined feature to obtain a predicted response result of the substance to be tested against the diseased cell.

20. A non-transitory computer-readable storage medium, having stored thereon computer program instructions that, when executed by a processor of an electronic device, cause the processor to perform a prediction method comprising:
according to a molecular structure of a substance to be tested, determining substance features of the substance to be tested;
performing feature extraction on a diseased cell of a target category to obtain at least one cell feature of the diseased cell; and
according to the substance features and the at least one cell feature, predicting a response result of the substance to be tested against the diseased cell.
